(19) United States
(12) Patent Application Publication
(10) Pub. No.: US 2017/0130272 A1
(43) Pub. Date: May 11, 2017

(54) A Diagnostic Marker For Paget's Disease

(71) Applicant: Consiglio Nazionale Delle Ricerche, Roma (IT)

(72) Inventor: Fernando Gianfrancesco, Fontegreca, Caserta (IT)

(73) Assignee: Consiglio Nazionale Delle Ricerche, Roma (IT)

(21) Appl. No.: 15/312,501

(22) PCT Filed: May 21, 2015
(86) PCT No.: PCT/IB2015/053731

§ 371 Date: Nov. 18, 2016

(30) Foreign Application Priority Data
May 21, 2014 (IT) TO2014A000404

(51) Int. Cl. C12Q 1/68; G01N 33/68; C07K 14/47; G01N 33/574

(52) U.S. Cl. CPC C12Q 1/6886; C12Q 1/6883; G01N 33/57407; G01N 33/6893; C07K 14/47; C12Q 2600/158; C12Q 2600/156; G01N 2800/50; G01N 2800/10

(57) ABSTRACT

The invention relates to an in vitro or in vivo method for diagnosing Paget's disease or bone tumors associated therewith or for determining the predisposition of a subject affected by Paget's disease of bone to develop a bone tumor, the method being based on the detection of a mutation in the nucleotide sequence of the ZNF687 gene or in the amino acid sequence of the protein encoded by the mutated ZNF687 gene. The invention also refers to isolated nucleic acids comprising the mutated nucleotide sequence of the ZNF687 gene or a portion thereof and the encoded amino acid sequences.

The present invention falls within the field of diagnostic markers.

More particularly, the present invention relates to a new diagnostic marker for Paget's disease of bone and bone tumors associated therewith, such as for example giant cell tumors (GCT) and osteosarcoma.

Paget's disease of bone is a metabolic disorder characterized by focal abnormalities in bone remodeling, which typically causes deformity and increased bone size in one or more areas of the skeleton (monostotic form or polyostotic form). In some cases, patients affected by Paget's disease of bone—and particularly those affected by its polyostotic form—incur into neoplastic degeneration of the affected bones (GCT), caused by uncontrolled proliferation of the spindle-shaped stromal cells.

The primary cell abnormality in Paget's disease of bone relates to osteoclasts, which show many functional and morphological abnormalities.

Paget's disease of bone is also characterized by specific modifications of the biochemical distribution of non-collagenous proteins in the bone matrix immediately adjacent to Haversian canals and by bone neoangiogenesis. Some of these modifications are very similar to those found in bone metastases in cancer patients and in patients affected by multiple myeloma.

Epidemiological data point out that Paget's disease of bone may be considered as a disease that predisposes to the appearance of primary bone neoplasms in subjects over 40 years old. The connection between Paget's disease of bone and an increased risk of malignant transformation of the affected bones has been described for the first time by Sir James Paget in his first paper on osteitis deformans in 1876. Since then, several literature papers reported a higher incidence of osteosarcomas and GCT in patients affected by Paget's disease of bone, and neoplastic degeneration of bones affected by Paget's disease of bone was estimated to occur in 1% of patients, with an increase in the risk as high as 30 times over that of the general population (Roodman 2001, Gebhart 1998).

In 2002, a few mutations were identified which affect the ubiquitination domain of the protein encoded by the SQSTM1 gene which are detected in 10% and 40% of cases of sporadic and familial Paget's disease of bone, respectively (Laurin et al 2002, Hocking et al 2002).

More recently, other genes (CSF1, OPTN, TNFRSF11A, PML, RIN3, NUP205, TM7SF4) have been associated with the Paget's disease of bone in patients without the mutation of SQSTM1 (Albagha et al 2010, Gianfrancesco et al 2012, Albagha et al 2011).

A familial clustering was also demonstrated in a few Paget's pedigrees affected by GCT and less frequently by osteosarcoma, particularly in patients of Italian origin (Rendina et al 2004, Gianfrancesco et al 2013, Wu et al 1991).

The International patent application WO2011/128646 describes a method of diagnosis for Paget's disease of bone, which comprises testing a sample from a subject for sequence changes at or within one or more chromosome loci selected from the group consisting of 10pl3, 1pl3.3, 18q21, 8q22.3, 7q33, 14q32.12, 15q24.1, 6p22.3 and Xq24.

However, the currently known diagnostic markers for Paget's disease of bone have the disadvantage of not being suitable for diagnosing the clinically serious forms of the disease, such as for example those with neoplastic degeneration of the affected bones. These families do not have mutations in the SQSTM1 gene.

Therefore, there is a need for a new diagnostic marker for Paget's disease of bone which overcomes the drawbacks of the prior art, which particularly allows for diagnosing and predicting the risk of developing bone tumors associated with Paget's disease of bone.

Such a need has now been met by the present inventors who identified a new diagnostic marker for Paget's disease of bone and/or bone tumors associated therewith, particularly osteosarcomas and giant cell tumors, which does not show the above-mentioned drawbacks.

Such a new marker is the ZNF687 gene, which encodes for the zinc finger protein 687, a component of the transcriptional coregulator Z3 complex.

By whole-exome sequencing and by examining a family including 14 members affected by Paget's disease of bone, four of which had developed GCT at Paget's sites, the present inventors detected a missense mutation in the ZNF687 gene (c.2810C>G, P937R). The sequencing of 7 non-related patients affected by GCT as a complication of Paget's disease of bone has allowed for identifying the same mutation in all of the patients, which suggests a founder effect. The same mutation and also a second missense mutation in the same gene (c.725G>T, p.Ser242Ile) were observed in patients with a serious form of Paget's disease of bone. Furthermore, in a non-pagetic patient, affected by GCT and with a family history of Paget's disease of bone (one parent affected) a third mutation was identified (c. 1994 C>T, P665L) in the ZNF687 gene.

The ZNF687 gene, which encodes for a member of the transcriptional regulation Z3 complex, is highly expressed during osteoblastogenesis and osteoclastogenesis, and is dramatically upregulated in tumor tissue derived from patients affected by giant cell tumor/Paget's disease of bone. ZNF687 knockdown results in a marked decrease in transcription of 2-IIIc receptor of FGF (FGFR2IIIc), revealing a tight correlation between these two genes. Moreover, by immunohistochemical analysis of patients affected by giant cell tumor/Paget's disease of bone, the inventors observed a strong FGFR2IIIc staining in multinucleated giant cells and stromal cells, which supports the role of FGFR2IIIc in the pathogenesis of these tumors.

These and other experimental findings, which will be described in detail in the experimental section that follows, allowed the inventors to conclude that the detection of mutations in the nucleotide sequence of the ZNF687 gene represents a useful diagnostic tool for Paget's disease of bone and bone tumors associated therewith, particularly giant cell tumors and osteosarcomas, as well as a useful prognostic tool for establishing the predisposition of a subject affected by Paget's disease of bone of developing a bone tumor associated therewith, particularly giant cell tumor or osteosarcoma.

The wild-type nucleotide sequence of the ZNF687 gene is illustrated in SEQ ID NO:1.

One aspect of the present invention is thus an in vitro or ex vivo method for diagnosing Paget's disease of bone or bone tumor, which comprises the step of determining, in a nucleic acid sample of a subject suspected of being affected by Paget's disease of bone or bone tumor, the presence of at least one mutation in the wild-type nucleotide sequence of the ZNF687 gene as represented by SEQ ID NO:1, the presence of said at least one mutation being indicative of Paget's disease of bone or bone tumor.

A further aspect of the present invention is an in vitro or ex vivo method for determining the predisposition of a subject affected by Paget's disease of bone to develop a bone tumor, which comprises the step of determining, in a nucleic acid sample of a subject affected by Paget's disease of bone, the presence of at least one mutation in the wild-type nucleotide sequence of the ZNF687 gene as represented by SEQ ID NO:1, the presence of said at least one mutation being indicative of a predisposition to develop a bone tumor.

In a preferred embodiment of both of the methods mentioned above, the bone tumor is a giant cell tumor or an osteosarcoma.

In another preferred embodiment, the mutation is a missense mutation.

Examples of missense mutations suitable for use in the methods of the invention are the nucleotide mutation C>G at nucleotide position 2810 of SEQ ID NO:4, the nucleotide mutation G>T at nucleotide position 725 of SEQ ID NO:2 and the nucleotide mutation C>T at nucleotide position 1994 of SEQ ID NO:3. Further mutations particularly suitable to be detected in the methods of the invention are mutations located in the coding region of the gene.

The mutations can be detected by any known method, for example by nucleic acid amplification reaction and detection of the amplification product or by sequencing the whole nucleotide sequence of the ZNF687 gene or a portion thereof and comparing it with the wild-type sequence of ZNF687, used as a known control sequence.

These are per se known methodologies and the application thereof to the methods of the invention is well within the ability of the person of ordinary skill in the art.

Another aspect of the invention is an isolated nucleic acid, preferably a genomic DNA, mRNA or cDNA, as defined in claim 1.

The isolated nucleic acid of the invention comprises the coding region of the ZNF687 gene and the mutation is located in said coding region. As previously indicated with reference to the methods of the invention, the mutation is a missense mutation, particularly the nucleotide mutation C>G at nucleotide position 2810 of SEQ ID NO:4 or the nucleotide mutation G>T at nucleotide position 725 of SEQ ID NO:2 or the nucleotide mutation C>T at nucleotide position 1994 of SEQ ID NO:3. Therefore, preferred isolated nucleic acids according to the invention are nucleic acids with the sequence SEQ ID NO:2 or SEQ ID NO:3 or SEQ ID NO:4.

Such nucleic acids are suitable for use in the manufacture of the corresponding mutated protein, by using recombinant DNA methodologies which are per se known and within the ability of the person of ordinary skill in the art.

A further aspect of the present invention is thus an expression vector including a nucleic acid as previously defined, as well as a host cell transformed with the said vector.

The host cell of the invention is capable of expressing the mutated ZNF687 protein, for instance the protein with the sequence SEQ ID NO:5 or SEQ ID NO:6 or SEQ ID NO:7.

Such mutated ZNF687 protein is in turn useful for the manufacture of specific antibodies, for example monoclonal or polyclonal, chimeric or humanized antibodies, which are manufactured by conventional methods, the accomplishment of which is within the ability of the person of ordinary skill in the art. The said antibodies are useful as assay reagents for carrying out immunodiagnostic methods within the scope of the present invention, such as for example ELISA immunoassay methods.

Therefore, another aspect of the present invention is an in vitro or ex vivo method for diagnosing Paget's disease of bone or bone tumor, which comprises the step of determining, in a biological sample from a subject suspected of being affected by Paget's disease of bone or bone tumor, the presence of at least one ZNF687 protein which is mutated compared to the wild-type ZNF687 protein sequence as represented by SEQ ID NO:8, the presence of said at least one mutated protein being indicative of Paget's disease of bone or bone tumor.

Still another aspect of the present invention is an in vitro or ex vivo method for determining the predisposition of a subject affected by Paget's disease of bone to develop a bone tumor, which comprises the step of determining, in a biological sample from a subject affected by Paget's disease of bone, the presence of at least one ZNF687 protein which is mutated compared to the wild-type ZNF687 protein sequence as represented by SEQ ID NO:8, the presence of said at least one mutated protein being indicative of a predisposition to develop a bone tumor.

In both of the above-mentioned methods, the mutated ZNF687 protein compared to the wild-type ZNF687 protein sequence represented by SEQ ID NO:8 is preferably selected from SEQ ID NO:5, SEQ ID NO:6 and SEQ ID NO:7.

The part that follows, related to Examples and Experimental section, is provided only by way of illustration and not as a limitation of the scope of the invention as defined in the appended claims.

### EXAMPLES

### 1. Experimental Section

Methods

Whole-Exome Sequencing Strategy.

The DNA from the four subjects belonging to the pedigree with GCT/PDB was used for exome sequencing by using the NimbleGen SeqCap EZ Exome TM capture kit (Roche) and the resulting fragments were sequenced by Illumina Genome Analyzer IIx (Illumina, San Diego, Calif., USA) with sequences of 90 bp in length. 58.458.230 sequences for sample V-4, 55.895.176 sequences for sample V-18, 45.223.865 sequences for sample V-11 and finally 50.109.914 sequences for sample V-7 were obtained. These sequences were aligned with human genomic sequence GRCh37/hg19 with the software MAQ7 and NextGENe v2.00. A coverage of over 45× for each sample, the sequencing of all the exons and the 5′ and 3′ untranslated regions were obtained. The analysis for identification of variations was carried out with the DNA bioinformatics platform Nexus. The variants obtained were filtered with those present in the SNP database (dbSNP131). Assuming the synonymous variants had less chances of being pathogenetic, the inventors only focused on non-synonymous variants (NS), mutations at splice donor and acceptor sites (SS), and small coding insertions or deletions (I).

Mutational Analysis.

The analysis for validation and segregation of the selected variants was performed by Sanger sequencing. PCR amplification and direct sequencing protocols have been described previously (Gianfrancesco et al. 2012). Oligonucleotides used for amplifying and sequencing the ZNF687 gene are:

exon 2A:
(SEQ ID NO: 9)
5′-CCTCCTCGTTCCTGTTTTCA-3′
(SEQ ID NO: 10)
5′-CAATGGTGGGGAAGAAACAG-3′
exon 2B:
(SEQ ID NO: 11)
5′-GGACCTGTTTGCTCATTTTG-3′
(SEQ ID NO: 12)
5′-AACTGTACGCTCACCACCTT-3′
exon 2C:
(SEQ ID NO: 13)
5′-CGGGAATATCACAAGGACTG-3′
(SEQ ID NO: 14)
5′-TATAGGCAGGGAGCAGGTTC-3′
exon 2D:
(SEQ ID NO: 15)
5′-AATGGTGCCTCGGTGGTGAT-3′
(SEQ ID NO: 16)
5′-GAGCATCATGGGGCAGGTTG-3′
exon 3:
(SEQ ID NO: 17)
5′-CATGCTTTCGCTGCCTGGAG-3′
(SEQ ID NO: 18)
5′-GAGGGATATAGCAGGGAAGA-3′
exons 4-5:
(SEQ ID NO: 19)
5′-CCTTTCTCCACCCTGCTCAT-3′
(SEQ ID NO: 20)
5′-ACCCATTCCCCATCCCTCTG-3′
exon 6:
(SEQ ID NO: 21)
5′-CCGTCTTGTCCTCTGCTCTT-3′
(SEQ ID NO: 22)
5′-AGGAGAGCAGGGGTAGATGT-3′
exons 7-8:
(SEQ ID NO: 23)
5′-GCCTGGCTCTGACATCTACC-3′
(SEQ ID NO: 24)
5′-CCTTACCACCACCCCTACTG-3′
exon 9:
(SEQ ID NO: 25)
5′-GCTGTGCTAGGGCTTTGAGT-3′
(SEQ ID NO: 26)
5′-AGGCAGCAGTAGGGAAAACC-3′

Expression Analysis

In order to analyze the expression pattern of the ZNF687 gene, a real-time PCR was carried out on total RNA derived from human adult tissues purchased from Stratagene with the Cycler DNA Engine Opticon 2 system (Biorad). One microgram of total RNA was reverse transcribed with the RevertAid RT kit (Thermo Scientific). The expressions were normalized to the HPRT gene to account for differences in the starting material and in the cDNA reaction efficiency. An electrophoresis on agarose gel was performed to further confirm the specificity of the PCR products. The oligonucleotides of the ZNF687 transcript (5′-AGGAGTCGTCTTCATCTTCA-3′ (SEQ ID NO:27) and 5′-TTCACTGACTTGCCATGCTC-3′ (SEQ ID NO:28) were used to generate a product of 214 bp. The HPRT oligonucleotides (5′-TGGCGTCGTGATTAGTGATG-3′ (SEQ ID NO:29) and 5′-GCACACAGAGGGCTACAATG-3′ (SEQ ID NO:30) were used to generate a product of 185 bp.

Cultures and Primary Cell Lines.

Cell lines MG63, RAW264.7, U2OS and HEK293 were plated on 60-mm plates using DMEM culture medium (Gibco) supplemented with 10% fetal bovine serum, 2 mM L-glutamine, 100 U/mL penicillin and 100 mg/mL streptomycin (Gibco) until they reached a 70-80% confluence.

siRNA Transfection

1.5×10⁵ cells were plated on 60-mm plates. 24 hours later, MG63 cells were transfected with a mixture of small interfering RNAs ZNF687 10 nM (siRNAs) Duplex Trilencer-27 (Origene) and 26.5 uL of IBAFECT reagent (Iba Solutions For Life Sciences) in OPTIMEM (1×)+ GlutaMax-I reduced serum media (Gibco, Life Technologies). Each transfection was performed in triplicate and repeated four times. 48 and 72 hours post-transfection, the cells were blocked to isolate RNA and proteins.

Protein Extraction and Western Blot Analysis for ZNF687 and FGFR2IIIc

After 48 and 72 hours of incubation, MG63, RAW264.7 and U2OS cells were lysed with lysis buffer (5 ml 100% glycerol, 50 μl tween20, 500 μl Triton 100, 3.5 ml 5M NaCl, 2.5 ml 1M Tris pH 7.9, 250 μl M MgCl₂, all in a final volume of 50 ml). The lysed cells were centrifuged at 14000 g for 5 minutes. Proteins were denatured by boiling in sample buffer, separated on 8% SDS-PAGE, transferred onto an Immun-Blot PVDF membrane for protein blotting (BIO-RAD) and blocked for 1 hour in 5% dried skimmed milk in TBS-T (10 mM Tris-HCl pH 7.5, 100 mM NaCl, 0.1% (v/v) Tween-20). Antibodies used for protein recognition are: anti-ZNF687 polyclonal rabbit antibody ab105544 (abcam 1:500), anti-Bek polyclonal rabbit antibody (c-17): sc-122 (Santacruz 1:100) and anti-B actin polyclonal mouse antibody (c4) sc-47778 (Santacruz 1:1000). Goat anti-rabbit antibodies conjugated with peroxidase (1:1.000 for ZNF687 protein and 1:2000 for FGFR2 protein and anti-mouse (diluted 1:15000 in TBST) (Biorad) were used as secondary antibodies.

The Western Blot assay was quantified with the Image J program.

Osteoclast Formation Assay

Peripheral blood mononuclear cells were separated by Ficoll density gradient centrifugation and the cells in suspension were plated in 24-well plates (3×10⁵). Osteoclasts were generated starting from cells cultured for 14-21 days in alpha-minimal essential medium (α-MEM) (SIGMA, Life Science) containing 10% fetal bovine serum (FBS), 2 mM L-glutamine, 100 U/ml penicillin and 100 mg/ml streptomycin (Gibco) as well as 25 ng/mL macrophage colony stimulating factor (M-CSF) (Peprotech, Rocky Hill, N.J., USA) and 30 ng/mL soluble human recombinant sRANK ligand (RANKL) (Peprotech, Rocky Hill, N.J., USA).

Results

Identification of the Gene Responsible for Giant Cell Tumor Associated with Paget's Disease.

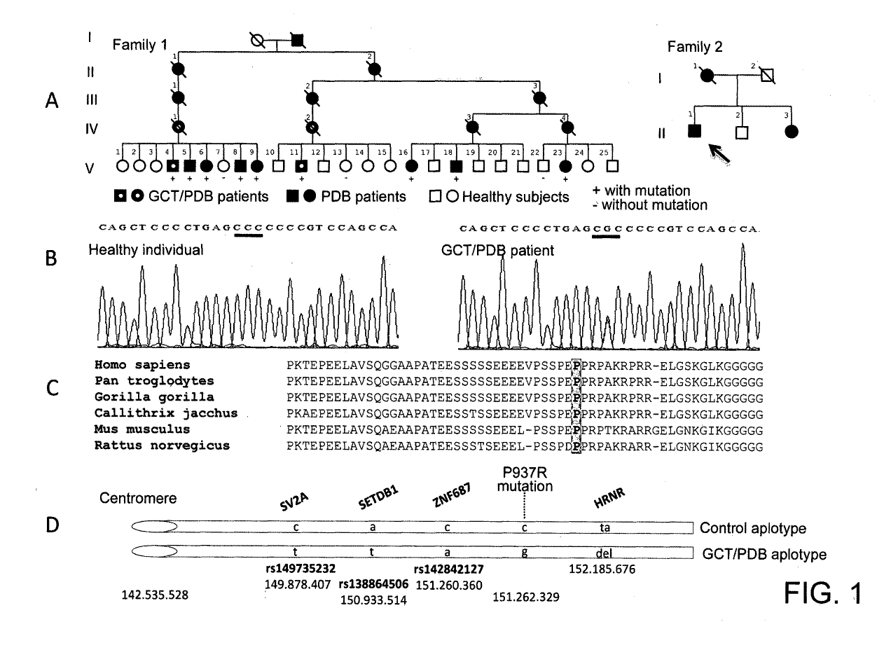

Recently, the inventors described a family with 14 individuals affected by Paget's disease, 4 of which with giant cell tumor (Gianfrancesco et al 2013). A whole-exome sequencing was carried out on the DNA of three patients affected by PDB (V-4, V-18, V-11), two of which with giant cell tumor, and of one healthy subject (V-7) (FIG. 1A). By adopting a selection scheme, the variants recorded in the SNP database (dbSNP131), and then those present in 4 exomes available at the inventors' laboratory and derived from 4 subjects affected by diseases that are not related with the tested one, were excluded. After such a filtering, the data from the three affected individuals were compared, with the exception of variants present in the healthy subject, identifying 57 variations in 45 candidate genes. Twenty-one of these variants were confirmed by Sanger direct sequencing in the three patients, yet only one of these showed a complete segregation with the disease phenotype in the family: c.2810C>G that determines the amino acid substitution P937R, in exon 6 of the ZNF687 gene located on chromosome 1 (1q21.3) in one of the linkage regions described in the previous work (Gianfrancesco et al 2013). The mutation is heterozygous in patients and is absent in non-affected individuals (FIG. 1B). Such a mutation has not been detected in a cohort of 564 control individuals from the same geographical area and the evolutionary analysis has shown that proline 937 is conserved in all mammals, suggesting its important biological role (FIG. 1C). To validate the results, the inventors analyzed the ZNF687 gene in 7 patients independently affected by GCT/PDB, 4 of which were from the same localized area of South Italy and three were Italian patients or American patients with Italian ancestors (Rendina et al 2006, Gennari et al 2010, Michou et al 2011). Interestingly, the same mutation c.2810C>G was detected in all GCT/PDB patients, suggesting they share a common founder mutation. To validate this hypothesis, the segregation of rare variants was assessed in genes surrounding mutation c.2810C>G, detecting heterozygous variations for SNPs rs149735232 (SV2A), rs138864506 (SETDB1), rs142842127 (ZNF687), of TA in position 152.185.676 of the HRNR gene. This aplotype was not found in any of the 559 tested controls of the same geographical area, suggesting that the mutation is located on a common aplotype which identifies all GCT/PDB patients (FIG. 1D).

Molecular Analysis of the ZNF687 Gene in Patients Affected by Paget's Disease without Neoplastic Degeneration.

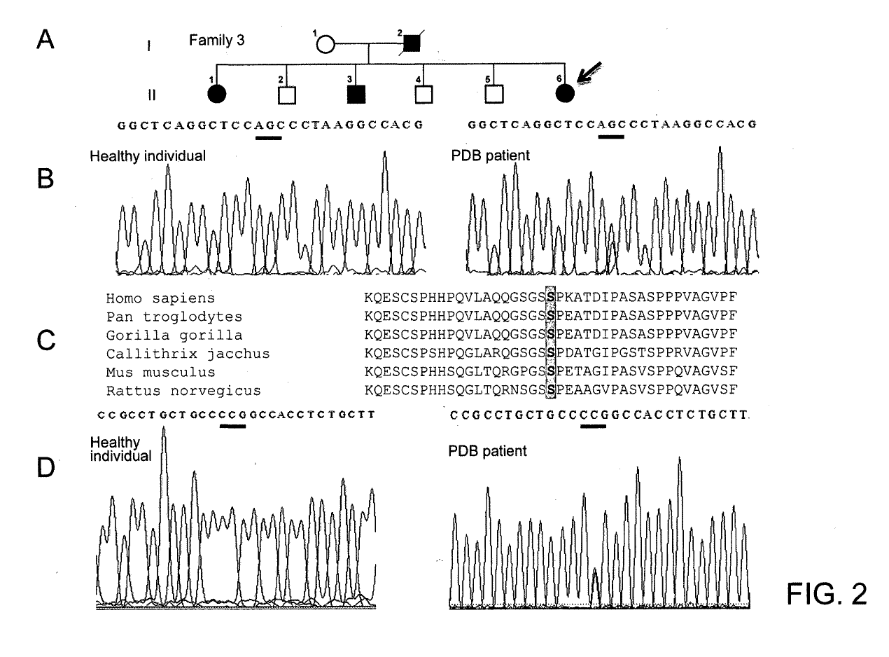

To verify the role of the ZNF687 gene in Paget's patients without neoplastic degeneration, the whole coding region of the gene, consisting of 9 exons, was analyzed in 22 of 37 Paget's subjects who had tested negative for mutations in the SQSTM1 gene. Each of the tested subjects had at least one other family member with the disease, confirming the autosomal dominant inheritance of the disease in these families. This analysis allowed for detecting in the tested subject of family 2 (FIG. 1A) the same mutation c.2810C>G (P937R) indicated above and a new mutation, c.725G>T (S242I) in exon 2 of the tested subject of family 3 (FIG. 2A). The p.P937R mutation segregates with the disease phenotype in family 2, it being present in patient II-3, but not in the healthy sibling II-2. Also the p.S242I mutation segregates with the disease phenotype, it being present in the two affected siblings (II-1, II-3, II-6) while it has not been detected in the non-affected members (II-2, II-4, II-5) (FIGS. 2A-2B). This mutation, which determines the substitution of an isoleucine for a serine in position 242 (p.S242I), is not reported in the database “1000 Genomes”. Serine in position 242 is highly conserved in mammals (FIG. 2C) and the substitution thereof is predicted as detrimental by the prediction programs SIFT, PolyPhen and Align GVGD. In order to verify the presence of mutations in the ZNF687 gene also in the GCT not related to the Paget's disease of bone (conventional GCT), the genetic analysis was performed in 44 patients affected by such disease, 38 of which had somatic mutations in the H3F3A gene. This analysis resulted in the identification of another mutation (c. 1994 C>T, P665L) in exon 2 of the ZNF687 gene (FIG. 2D) in a patient whose anamnesis had not shown a family history of Paget's disease of bone.

The c.2810C>G mutation (p.937R) was assessed by the Taqman assay in two cohorts of Paget's patients of different ethnic groups. It has been identified in 2 Italian patients out of 628 and in 2 patients out of 339 of the multi-ethnic cohort (Michou et al 2011). Moreover, this mutation was not detected in 564 control individuals of Italian origin and in 269 control individuals of American origin. All the patients bearing the mutation in the ZNF687 gene showed a very serious form of the disease, predicting that these patients are predisposed to development of neoplastic complications and that the pharmacological treatment prevents the onset thereof. In fact, all GCT/PDB cases show a decreased response to treatment with bisphosphonates and an active persistency of the disease, with ALP levels well over the normal value range. Together, these data suggest that the persistency of an active Paget's disease may increase the risk of GCT complications, at least in genetically predisposed subjects.

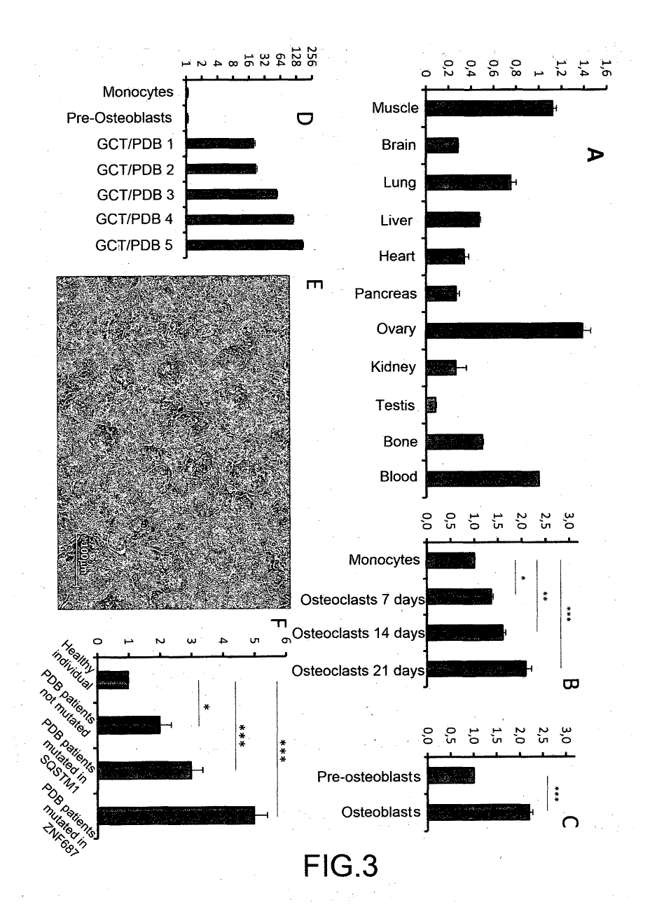

Expression of the ZNF687 gene increases during the differentiation of osteoclasts and osteoblasts. The expression profile of the ZNF687 gene was analyzed by using a panel of RNAs extracted from human adult tissues through a real-time PCR assay, revealing its expression in all the examined tissues (FIG. 3A). The expression of the gene was also analyzed during osteoclastogenesis induced on peripheral blood monocytes through treatment with MCSF and RANKL. Expression of ZNF687 increases significantly during the differentiation process (FIG. 3B). This result was also confirmed during the differentiation process of osteoclasts induced in murine cells. Expression of ZNF687 mRNA also increases in differentiated osteoblasts compared to their undifferentiated precursors (FIG. 3C). These data suggest a role for ZNF687 during differentiation of osteoclasts and osteoblasts, showing its involvement in the regulation of genes implicated in these two processes. The giant cell tumor that develops on pagetic bone is the consequence of a modification of both processes. Indeed, in RNA extracted from the giant cell tumor of pagetic patients, which includes the giant cells derived from osteoclasts and the stromal cells of osteoblastic origin, the inventors found a significant increase in ZNF687 expression (FIG. 3D). Finally, through immunohistochemical techniques, by using specific antibodies against the ZNF687 protein, a very intense staining was observed in the nucleus of the tumor giant cells in GCT/PDB patients compared to PDB patients (FIG. 3E).

By Real Time PCR, the expression level of ZNF687 in the peripheral blood of PDB patients without mutations in the SQSTM1, PDB patients with mutations in the SQSTM1 gene and PDB patients with mutations in the ZNF687 gene, was also evaluated. Interestingly, the ZNF687 expression levels are significantly higher in PDB patients with or without a SQSTM1 mutation compared to healthy control subjects (FIG. 3F). Furthermore, a greater increase (5-folds) was observed in PDB patients with ZNF687 mutations. These results suggest that ZNF687 is involved in the ethiopathogenesis of Paget's disease of bone, independently of the patients' genetic background. Its expression profile considerably correlates with the clinical seriousness of the disease.

ZNF687 Regulates the Expression of the Receptor FGFR2.

Recently, a transcriptional coregulating complex designated as Z3, formed by proteins ZMYND8, ZNF687 and ZNF592, was identified and its interaction with the ERα transcription factor was defined (Malovannaya et al 2011). Bioinformatic analyses allowed for hypothesizing that the expression of the FGFR2 gene, which has in its genomic region a binding site for the Z3 complex (recognized in Chip-Seq experiments by ZMYND8 antibody), may be regulated thereby. ZNF687 was silenced by use of a specific siRNA in cell lines MG63 (osteoblast line) and RAW264.7 (osteoclast line). 72 hours later, a decrease in FGFR2IIIc was observed both at the mRNA and the protein level in both the cell lines. These results point out that FGFR2IIIc is regulated by the Z3 complex. As for the ZNF687 gene, an increase was also observed in the expression of FGFR2 during cell differentiation of osteoclasts and osteoblasts. Finally, in sections of tumor tissue of GCT/PDB patients, an increase in the expression of the FGFR2 protein, apart from in mesenchymal stromal cells, was also confirmed in multinucleated giant cells, supporting its role in the pathogenesis of this tumor.

### 2. Diagnostic Kit According to the Invention—Assay by Nucleotide Sequencing

An example of a diagnostic kit for identifying, by nucleotide sequencing, the causative mutation c.2810C>G (P937R) in exon 6 of the ZNF687 gene comprises:
a pair of oligonucleotides (sense 5′-CCGTCTTGTCCTCTGCTCTT-3′ (SEQ ID NO:21), anti-sense 5′-AGGAGAGCAGGGGTAGATGT-3′ (SEQ ID NO:22) specific for the exponential amplification reaction of a fragment of the human ZNF687 gene where the causative mutation is located;
a single oligonucleotide (sense 5′-CCGTCTTGTCCTCTGCTCTT-3′ (SEQ ID NO:21) specific for the sequencing of the sense strand of the amplified fragment;
a single oligonucleotide (anti-sense 5′-AGGAGAGCAGGGGTAGATGT-3′ (SEQ ID NO:22) specific for the sequencing of the anti-sense strand of the amplified fragment;
a control DNA from a healthy individual not bearing the test mutation;
a 5× master mix (5 times more concentrated than the working reaction) comprising: 1 mM of a mixture of the four deoxynucleotides (dATP, dCTP, dTTP, dGTP), 5× reaction buffer (100 mM Tris, 50 mM Hepes, 12.5 mM Magnesium Sulfate, 50 mM Potassium Chloride and 50 mM Ammonium Sulfate) and 5 Units of Taq polymerase.

Amplification Protocol.

In a final volume of 25 microlitres, 100 ng of genomic DNA are combined with 0, 5 microM of each oligonucleotide, 5 microlitres of 5× Master mix and H₂O.

The exponential amplification reaction comprises a first cycle of 3 minutes at 94° C. for the denaturation of the genomic DNA, followed by 40 cycles that comprise: 30 seconds at 94° C., 30 seconds at 62° C., 45 seconds at 72° C.; a final cycle at 72° C. for 10 minutes. The PCR product can be visualized on a 1.5% agarose gel.

Sequencing

The amplification product is purified with the enzyme exosap and sequenced in a final volume of 20 microlitres with 2 micromolar oligonucleotide F or R and the Dye Terminator Ready Reaction Kit (Applied Biosystem). The reactions are carried out for 25 cycles that comprise: one step at 95° C. for 10 sec, 55° C. for 5 sec and 60° C. for 2 min. The sequencing reactions are purified and separated on ABI prism 3700 Genetic Analyser (Applied Biosystems).

### 3. Diagnostic Kit According to the Invention—Assay by the TaqMan Method

The diagnostic kit for identifying the causative mutation c.2810C>G (P937R) in exon 6 of the ZNF687 gene comprises:
a pair of oligonucleotides specific for the exponential amplification reaction of a fragment of the human ZNF687 gene where the causative mutation is located;
a forward (F) oligonucleotide probe specific for the mutated base associated with a reporter fluorochrome at the 5′ end.

Taqman Genotyping Assay

The diagnostic kit for identifying by the TaqMan method the causative mutation c.2810C>G in exon 6 of the ZNF687 gene comprises:

Two TaqMan® MGB probes, of which:
one probe labeled with the fluorochrome VIC® for recognition of the Allele 1 sequence (wild type);
one probe labeled with the fluorochrome FAM™ for recognition of Allele 2 (mutated);
two sequence-specific sense and anti-sense oligonucleotides for amplifying the sequence of interest;
Master Mix TaqMan® Universal PCR UP (Ultra Pure) containing: AmpliTaq Gold® DNA polymerase (1×), the fluorochrome ROX™ as a passive internal reference for assuring a better analysis on the real-time PCR tools, a commercially available reaction buffer and a mixture of the four deoxynucleotides (dATP, dCTP, dTTP, dGTP).

Amplification Protocol

In a final volume of 25 μL, 1-20 ng of genomic DNA are combined with 12.5 μL of TaqMan Universal PCR Master Mix (2×), 1.25 μL of the mixture of oligonucleotides and sequence-specific probes (20×) and sterile water to reach the final volume.

Exponential Amplification Reaction

A first cycle of 10 minutes at 95° C. is carried out for activating AmpliTaq Gold® DNA Taq polymerase, followed by 40 cycles that comprise: 15 seconds at 92° C. for the denaturation of the genomic DNA and 1 minute at 60° C. for assuring the annealing of the oligonucleotides and the sequence-specific probes and the subsequent extension of the fragment of interest. By using the thermocycler Applied Biosystems 7900HT Fast Real-Time PCR System, amplification curves are generated which may then be analyzed through the SDS Software v2.2.2 or subsequently for the 7900HT Fast System.

### REFERENCES

Roodman G D. Studies in Paget's disease and their relevance to oncology. Semin Oncol, 2001; 15-21.
Gebhart M, Vandeweyer E, Nemec E. Paget's disease of bone complicated by Giant Cell Tumor. Clin Orthop Relat Res. 1998; 352:187-93.
Laurin N, Brown J P, Morissette J, Raymond V. Recurrent mutation of the gene encoding sequestosome 1 (SQSTM1/p62) in Paget disease of bone. Am J Hum Genet 2002; 70:1582-88.
Hocking L J, Lucas G J, Daroszewska A, et al. Domain specific mutations in Sequestosome 1 (SQSTM1) cause familial and sporadic Paget's disease. Hum Mol Genet 2002; 11:2735-39.
Albagha O M, Visconti M R, Alonso N, Langston A L, Cundy T, Dargie R, Dunlop M G, Fraser W D, Hooper M J, Isaia G, Nicholson G C, del Pino Montes J, Gonzalez-Sarmiento R, di Stefano M, Tenesa A, Walsh J P, Ralston S H. Genome-wide association study identifies variants at CSF1, OPTN and TNFRSF11A as genetic risk factors for Paget's disease of bone. Nat Genet 2010; 42:520-4.
Albagha O M, Wani S E, Visconti M R, Alonso N, Goodman K, Brandi M L, Cundy T, Chung P Y, Dargie R, Devogelaer J P, Falchetti A, Fraser W D, Gennari L, Gianfrancesco F, Hooper M J, Van Hul W, Isaia G, Nicholson G C, Nuti R, Papapoulos S, Montes Jdel P, Ratajczak T, Rea S L, Rendina D, Gonzalez-Sarmiento R, Di Stefano M, Ward L C, Walsh J P, Ralston S H; Genetic Determinants of Paget's Disease (GDPD) Consortium. Genome-wide association identifies three new susceptibility loci for Paget's disease of bone. Nat Genet 2011; 43:685-9.
Gianfrancesco F, Rendina D, Di Stefano M, Mingione A, Esposito T, Merlotti D, Gallone S, Magliocca S, Goode A, Formicola D, Morello G, Layfield R, Frattini A, De Filippo G, Nuti R, Searle M, Strazzullo P, Isaia G, Mossetti G, Gennari L. A nonsynonymous TNFRSF11A variation increases NFκB activity and the severity of Paget's disease. J Bone Miner Res 2012; 27:443-52.
Rendina D, Mossetti G, Soscia E, Sirignano C, Insabato L, Viceconti R, Ignarra R, Salvatore M, Nunziata V. Giant cell tumor and Paget's disease of bone in one family: geographic clustering. Clin Orthop Relat Res 2004; 421:218-24.
Gianfrancesco F, Rendina D, Merlotti D, Esposito T, Amyere M, Formicola D, Muscariello R, De Filippo G, Strazzullo P, Nuti R, Vikkula M, Gennari L. Giant cell tumor occurring in familial Paget's disease of bone: report of clinical characteristics and linkage analysis of a large pedigree. J Bone Miner Res. 2013; 28:341-50.
Wu R K, Trumble T E, Ruwe P A. Familial incidence of Paget's disease and secondary osteogenic sarcoma. A report of three cases from a single family. Clin Orthop Relat Res. 1991; 265:306-9.
Malovannaya A, Lanz R B, Jung S Y, Bulynko Y, Le N T, Chan D W, Ding C, Shi Y, Yucer N, Krenciute G, Kim B J, Li C, Chen R, Li W, Wang Y, O'Malley B W, Qin J. Analysis of the Human Endogenous Coregulator Complexome. Cell 2011; 145:787-799.
Rendina D, Gennari L, De Filippo G, Merlotti D, de Campora E, Fazioli F, Scarano G, Nuti R, Strazzullo P, Mossetti G. Evidence for increased clinical severity of familial and sporadic Paget's disease of bone in Campania, southern Italy. J Bone Miner Res 2006; 21:1828-35.
Gennari L, Gianfrancesco F, Di Stefano M, Rendina D, Merlotti D, Esposito T, Gallone S, Fusco P, Rainero I, Fenoglio P, Mancini M, Martini G, Bergui S, De Filippo G, Isaia G, Strazzullo P, Nuti R, Mossetti G. SQSTM1 gene analysis and gene-environment interaction in Paget's disease of bone. J Bone Miner Res 2010; 25:1375-84.
Michou L, Morissette J, Gagnon E R, Marquis A, Dellabadia M, Brown J P, Siris E S. Novel SQSTM1 mutations in patients with Paget's disease of bone in an unrelated multiethnic American population. Bone 2011; 48:456-60.

1. An isolated nucleic acid which comprises the whole nucleotide sequence of the ZNF687 gene or a portion thereof, wherein said whole nucleotide sequence of the ZNF687 gene or a portion thereof comprises at least one mutation with respect to the wild-type nucleotide sequence of the ZNF687 gene as represented by SEQ ID NO:1, characterized in that the mutation is selected from the group consisting of:
C>G at nucleotide position 2810 of SEQ ID NO:4,
G>T at nucleotide position 725 of SEQ ID NO:2, and
C>T at nucleotide position 1994 of SEQ ID NO:3.

2. The isolated nucleic acid according to claim 1, comprising the nucleotide sequence SEQ ID NO:2, SEQ ID NO:3 or SEQ ID NO:4.

3. The isolated nucleic acid according to claim 1, which is a genomic DNA, an mRNA or a cDNA.

4. An oligonucleotide or a probe which comprises from 10 to 30 consecutive nucleotides of the nucleic acid sequence according to claim 1 and which includes said mutation.

5. An expression vector which comprises a nucleic acid sequence according to claim 1.

6. A host cell which comprises the expression vector according to claim 5.

7. An in vitro or ex vivo method for diagnosing Paget's disease of bone or bone tumor, which comprises the step of determining, in a nucleic acid sample of a subject suspected of being affected by Paget's disease of bone or bone tumor, the presence of at least one mutation in the wild-type nucleotide sequence of the ZNF687 gene as represented by SEQ ID NO:1, the presence of said at least one mutation being indicative of Paget's disease of bone or bone tumor.

8. An in vitro or ex vivo method for determining the predisposition of a subject affected by Paget's disease of bone to develop a bone tumor, which comprises the step of determining, in a nucleic acid sample of a subject affected by Paget's disease of bone, the presence of at least one mutation in the wild-type nucleotide sequence of the ZNF687 gene as represented by SEQ ID NO:1, the presence of said at least one mutation being indicative of a predisposition to develop a bone tumor.

9. The method according to claim 7, wherein said bone tumor is a giant cell tumor or an osteosarcoma.

10. The method according to claim 7, wherein said mutation is located in the ZNF687 gene coding region.

11. The method according to claim 7, wherein said mutation is a missense mutation.

12. The method according to claim 11, wherein the missense mutation is selected from the group consisting of:
C>G at nucleotide position 2810 of SEQ ID NO:4,
G>T at nucleotide position 725 of SEQ ID NO:2, and
C>T at nucleotide position 1994 of SEQ ID NO:3.

13. The method according to claim 7, wherein the mutation is detected by nucleic acid amplification reaction and detection of the amplification product.

14. The method according to claim 7, wherein the mutation is detected by sequencing the whole nucleotide sequence of the ZNF687 gene or a portion thereof.

15. An isolated mutated ZNF687 protein,
characterized in that it is encoded by a nucleic acid according to claim 1.

16. An isolated mutated ZNF687 protein, which consists of the amino acid sequence selected from the group consisting of SEQ ID NO:5, SEQ ID NO:6 and SEQ ID NO:7.

17. An in vitro or ex vivo method for diagnosing Paget's disease of bone or bone tumor, which comprises the step of determining, in a biological sample from a subject suspected of being affected by Paget's disease of bone or bone tumor, the presence of at least one ZNF687 protein which is mutated compared to the wild-type ZNF687 protein sequence as represented by SEQ ID NO:8, the presence of said at least one mutated protein being indicative of Paget's disease of bone or bone tumor.

18. An in vitro or ex vivo method for determining the predisposition of a subject affected by Paget's disease of bone to develop a bone tumor, which comprises the step of determining, in a biological sample from a subject affected by Paget's disease of bone, the presence of at least one mutated ZNF687 protein with respect to the wild-type ZNF687 protein sequence as represented by SEQ ID NO:8, the presence of said at least one mutated protein being indicative of a predisposition to develop a bone tumor.

19. The method according to claim 17, wherein said bone tumor is a giant cell tumor or an osteosarcoma.

20. The method according to claim 17, which comprises contacting said biological sample with an antibody which is capable of specifically recognizing an isolated mutated ZNF687 protein encoded by a nucleic acid which comprises the whole nucleotide sequence of the ZNF687 gene or a portion thereof, wherein said whole nucleotide sequence of the ZNF687 gene or a portion thereof comprises at least one mutation with respect to the wild-type nucleotide sequence of the ZNF687 gene as represented by SEQ ID NO:1, characterized in that the mutation is selected from the group consisting of:
C>G at nucleotide position 2810 of SEQ ID NO:4,
G>T at nucleotide position 725 of SEQ ID NO:2, and
C>T at nucleotide position 1994 of SEQ ID NO:3.

21. (canceled)

22. The method according to claim 8, wherein said bone tumor is a giant cell tumor or an osteosarcoma.

23. The method according to claim 8, wherein said mutation is located in the ZNF687 gene coding region.

24. The method according to claim 9, wherein said mutation is located in the ZNF687 gene coding region.

25. The method according to claim 9, wherein said mutation is a missense mutation.

26. The method according to claim 8, wherein the mutation is detected by nucleic acid amplification reaction and detection of the amplification product.

27. The method according to claim 8, wherein the mutation is detected by sequencing the whole nucleotide sequence of the ZNF687 gene or a portion thereof.

28. The method according to claim 18, wherein said bone tumor is a giant cell tumor or an osteosarcoma.

29. The method according to claim 18, which comprises contacting said biological sample with an antibody which is capable of specifically recognizing an isolated mutated ZNF687 protein encoded by a nucleic acid which comprises the whole nucleotide sequence of the ZNF687 gene or a portion thereof, wherein said whole nucleotide sequence of the ZNF687 gene or a portion thereof comprises at least one mutation with respect to the wild-type nucleotide sequence of the ZNF687 gene as represented by SEQ ID NO:1, characterized in that the mutation is selected from the group consisting of:
C>G at nucleotide position 2810 of SEQ ID NO:4,
G>T at nucleotide position 725 of SEQ ID NO:2, and
C>T at nucleotide position 1994 of SEQ ID NO:3.


SEQUENCE LISTING (30 sequences)

SEQ ID NO: 1 - DNA, 3714 bp, Homo sapiens
atgggggatatgaagacccctgattttgatgacctccttgctgcctttgacatccctgacattgatgcgaatgaagccatccattctgggccagaagaaaatgaggggcctggaggcccagggaagccagaaccaggtgtaggaagtgaatctgaagacacagcagcagcctctgctggggatggccctggagttccagcccaggcctctgaccatggcctgccaccgccagacatttctgtagtcagtgtcattgtcaagaacactgtgtgtcccgagcagtctgaggccctggctggaggctcagcaggagacggggcccaggctgctggggtaactaaagaagggcctgtggggcctcatcgaatgcagaatggttttgggagccctgaaccttccctcccaggaactccccactctcctgctcctcccagtgggggcacctggaaagaaaaaggcatggaaggcaaaactcccttggacctgtttgctcattttggccctgagccaggggaccactcagatccgctgcctccctctgcaccctctcccactcgggagggggctctgaccccgcctcctttcccctcttcctttgagctggcccaggagaatggcccaggcatgcagccacctgtttcttccccaccattgggggccttgaagcaggagagctgcagcccccatcatccccaggtcctagcccaacaaggctcaggctccagccctaaggccacggacatccctgccagtgcctcgcctcccccagttgctggggtgcccttcttcaagcagtctccagggcaccagagccctcttgcctcccccaaagtgcccgtctgtcagcccttgaaggaagaagatgatgatgaggggccagtggacaagtcttccccaggaagtccccagagtccctctagtggggccgaggctgcagatgaggacagcaatgactcccctgcctccagctcctctaggcctcttaaggtgcggatcaagaccattaaaacatcctgcgggaatatcacaaggactgtaactcaggtcccctcagatcctgatccacctgcccccttggctgagggggccttcttggctgaggctagcctcttgaagctgtcccctgcaacacctacttctgagggtccaaaggtggtgagcgtacagttgggtgatggtacaaggctgaaaggcactgtgctgcctgtggccaccatccagaacgccagtactgccatgctgatggcagccagtgtggctcgcaaggctgtggtgctgcctggggggactgccaccagccctaagatgattgctaagaacgtgctaggcctggtgccccaagccctgcctaaggctgacgggcgggcagggctggggactgggggacagaaggtgaatggtgcctcggtggtgatggtgcaaccttcaaagacagctactgggccaagtacagggggcggcacagtgatatcacggacccagtccagcctggtggaggccttcaacaagatcctcaacagcaagaacctgctccctgcctataggccaaacctgagcccaccagctgaggctgggctggccctgcctcccaccggctaccgctgcctggagtgtggggatgccttctcattggagaagagcctggcacggcactatgaccgtcggagcatgcgcatcgaggtcacctgcaaccactgcgcccgccgcctggtcttcttcaacaagtgcagcctgctcctgcatgcacgtgaacacaaggacaaggggctcgtcatgcagtgctcacatttggtcatgaggcctgtagcccttgaccagatggtggggcagccggacatcacaccgctgctgcctgtagctgtcccacctgtctctggacctctggccttgcctgccttgggcaagggtgagggggccatcacctcctctgccattactacagttgctgctgaggcccctgtcctgccgctctccacagagccgcctgctgccccggccacctctgcttacacatgctttcgctgcctggagtgcaaggaacagtgccgggacaaggctggcatggcagctcacttccagcagctcggcccccctgcccctggggccaccagcaatgtgtgcccaacctgccccatgatgctccccaatcgctgcagcttcagcgcccaccagcgcatgcataagaatcgacccccccatgtctgtcctgagtgtgggggcaacttcctgcaagccaattttcagacccatctccgggaggcctgtctgcacgtctctcgccgtgtaggatacaggtgccccagctgttcagtggtgtttgggggtgtgaactccatcaagtcccacatccagacgtcgcactgcgaggttttccacaagtgccccatctgccccatggccttcaagtctgggccaagtgcccatgcccacctctactcccagcatcccagcttccaaactcagcaggccaagctgatctacaagtgcgccatgtgcgacacagtcttcactcacaaacccctcctctcctcacacttcgaccagcacttgctgccccagcgtgtcagtgtctttaagtgcccgtcttgtcctctgctctttgcccaaaaaaggaccatgctggaacatctcaagaacacccatcagtctgggcgcttggaggagactgctgggaaaggggccgggggtgccctgctgacccccaagactgagcctgaggagctggctgtttctcagggaggggcagcccctgctactgaggagtcgtcttcatcttcagaagaggaggaagtacccagctcccctgagcccccccgtccagccaaacggcctcggcgggaactagggagcaaaggcctcaagggtgggggtggggggcctggaggctggacctgtggcctgtgtcactcctggttccctgagcgtgatgaatacgtggcccacatgaagaaggagcatggcaagtcagtgaaaaagttcccctgtcgcctgtgtgagcgctccttctgctccgcccccagcctgaggcgccatgtcagagttaatcacgagggcatcaagcgagtttacccctgcaggtattgcacagagggaaaacgcaccttcagcagccgcctgatcctagagaaacatgtccaggtccggcacggcttgcagcttggggcccagtcccctggccgggggaccaccttggctcggggttccagtgccagagcccaggggccaggtcggaaacgccgccagtcttctgactcttgcagtgaggagcctgacagcacgacaccgccagccaagtcccccaggggcggacctggatctggaggccatggccctctgcgctaccggagcagcagctccacagaacagagcctcatgatggggttgagggtggaggatggtgcccagcagtgcctcgactgtggcttgtgctttgcctcccctggctccctgagccgacaccgtttcatcagccacaagaagagacggggtgtgggtaaagccagtgccctggggctgggggatggggaggaagaggcccctccatcaaggtctgaccccgatggtggagactcacccctgcctgcttctggaggcccactgacctgtaaggtctgtggcaagagctgcgacagccctctaaacctcaagacccacttccgcacgcatggcatggcgttcatcagggctcggcagggggctgttggggacaactag

SEQ ID NO: 2 - DNA, 3714 bp, Homo sapiens
atgggggatatgaagacccctgattttgatgacctccttgctgcctttgacatccctgacattgatgcgaatgaagccatccattctgggccagaagaaaatgaggggcctggaggcccagggaagccagaaccaggtgtaggaagtgaatctgaagacacagcagcagcctctgctggggatggccctggagttccagcccaggcctctgaccatggcctgccaccgccagacatttctgtagtcagtgtcattgtcaagaacactgtgtgtcccgagcagtctgaggccctggctggaggctcagcaggagacggggcccaggctgctggggtaactaaagaagggcctgtggggcctcatcgaatgcagaatggttttgggagccctgaaccttccctcccaggaactccccactctcctgctcctcccagtgggggcacctggaaagaaaaaggcatggaaggcaaaactcccttggacctgtttgctcattttggccctgagccaggggaccactcagatccgctgcctccctctgcaccctctcccactcgggagggggctctgaccccgcctcctttcccctcttcctttgagctggcccaggagaatggcccaggcatgcagccacctgtttcttccccaccattgggggccttgaagcaggagagctgcagcccccatcatccccaggtcctagcccaacaaggctcaggctccatccctaaggccacggacatccctgccagtgcctcgcctcccccagttgctggggtgcccttcttcaagcagtctccagggcaccagagccctcttgcctcccccaaagtgcccgtctgtcagcccttgaaggaagaagatgatgatgaggggccagtggacaagtcttccccaggaagtccccagagtccctctagtggggccgaggctgcagatgaggacagcaatgactcccctgcctccagctcctctaggcctcttaaggtgcggatcaagaccattaaaacatcctgcgggaatatcacaaggactgtaactcaggtcccctcagatcctgatccacctgcccccttggctgagggggccttcttggctgaggctagcctcttgaagctgtcccctgcaacacctacttctgagggtccaaaggtggtgagcgtacagttgggtgatggtacaaggctgaaaggcactgtgctgcctgtggccaccatccagaacgccagtactgccatgctgatggcagccagtgtggctcgcaaggctgtggtgctgcctggggggactgccaccagccctaagatgattgctaagaacgtgctaggcctggtgccccaagccctgcctaaggctgacgggcgggcagggctggggactgggggacagaaggtgaatggtgcctcggtggtgatggtgcaaccttcaaagacagctactgggccaagtacagggggcggcacagtgatatcacggacccagtccagcctggtggaggccttcaacaagatcctcaacagcaagaacctgctccctgcctataggccaaacctgagcccaccagctgaggctgggctggccctgcctcccaccggctaccgctgcctggagtgtggggatgccttctcattggagaagagcctggcacggcactatgaccgtcggagcatgcgcatcgaggtcacctgcaaccactgcgcccgccgcctggtcttcttcaacaagtgcagcctgctcctgcatgcacgtgaacacaaggacaaggggctcgtcatgcagtgctcacatttggtcatgaggcctgtagcccttgaccagatggtggggcagccggacatcacaccgctgctgcctgtagctgtcccacctgtctctggacctctggccttgcctgccttgggcaagggtgagggggccatcacctcctctgccattactacagttgctgctgaggcccctgtcctgccgctctccacagagccgcctgctgccccggccacctctgcttacacatgctttcgctgcctggagtgcaaggaacagtgccgggacaaggctggcatggcagctcacttccagcagctcggcccccctgcccctggggccaccagcaatgtgtgcccaacctgccccatgatgctccccaatcgctgcagcttcagcgcccaccagcgcatgcataagaatcgacccccccatgtctgtcctgagtgtgggggcaacttcctgcaagccaattttcagacccatctccgggaggcctgtctgcacgtctctcgccgtgtaggatacaggtgccccagctgttcagtggtgtttgggggtgtgaactccatcaagtcccacatccagacgtcgcactgcgaggttttccacaagtgccccatctgccccatggccttcaagtctgggccaagtgcccatgcccacctctactcccagcatcccagcttccaaactcagcaggccaagctgatctacaagtgcgccatgtgcgacacagtcttcactcacaaacccctcctctcctcacacttcgaccagcacttgctgccccagcgtgtcagtgtctttaagtgcccgtcttgtcctctgctctttgcccaaaaaaggaccatgctggaacatctcaagaacacccatcagtctgggcgcttggaggagactgctgggaaaggggccgggggtgccctgctgacccccaagactgagcctgaggagctggctgtttctcagggaggggcagcccctgctactgaggagtcgtcttcatcttcagaagaggaggaagtacccagctcccctgagcccccccgtccagccaaacggcctcggcgggaactagggagcaaaggcctcaagggtgggggtggggggcctggaggctggacctgtggcctgtgtcactcctggttccctgagcgtgatgaatacgtggcccacatgaagaaggagcatggcaagtcagtgaaaaagttcccctgtcgcctgtgtgagcgctccttctgctccgcccccagcctgaggcgccatgtcagagttaatcacgagggcatcaagcgagtttacccctgcaggtattgcacagagggaaaacgcaccttcagcagccgcctgatcctagagaaacatgtccaggtccggcacggcttgcagcttggggcccagtcccctggccgggggaccaccttggctcggggttccagtgccagagcccaggggccaggtcggaaacgccgccagtcttctgactcttgcagtgaggagcctgacagcacgacaccgccagccaagtcccccaggggcggacctggatctggaggccatggccctctgcgctaccggagcagcagctccacagaacagagcctcatgatggggttgagggtggaggatggtgcccagcagtgcctcgactgtggcttgtgctttgcctcccctggctccctgagccgacaccgtttcatcagccacaagaagagacggggtgtgggtaaagccagtgccctggggctgggggatggggaggaagaggcccctccatcaaggtctgaccccgatggtggagactcacccctgcctgcttctggaggcccactgacctgtaaggtctgtggcaagagctgcgacagccctctaaacctcaagacccacttccgcacgcatggcatggcgttcatcagggctcggcagggggctgttggggacaactag

SEQ ID NO: 3 - DNA, 3714 bp, Homo sapiens
atgggggatatgaagacccctgattttgatgacctccttgctgcctttgacatccctgacattgatgcgaatgaagccatccattctgggccagaagaaaatgaggggcctggaggcccagggaagccagaaccaggtgtaggaagtgaatctgaagacacagcagcagcctctgctggggatggccctggagttccagcccaggcctctgaccatggcctgccaccgccagacatttctgtagtcagtgtcattgtcaagaacactgtgtgtcccgagcagtctgaggccctggctggaggctcagcaggagacggggcccaggctgctggggtaactaaagaagggcctgtggggcctcatcgaatgcagaatggttttgggagccctgaaccttccctcccaggaactccccactctcctgctcctcccagtgggggcacctggaaagaaaaaggcatggaaggcaaaactcccttggacctgtttgctcattttggccctgagccaggggaccactcagatccgctgcctccctctgcaccctctcccactcgggagggggctctgaccccgcctcctttcccctcttcctttgagctggcccaggagaatggcccaggcatgcagccacctgtttcttccccaccattgggggccttgaagcaggagagctgcagcccccatcatccccaggtcctagcccaacaaggctcaggctccagccctaaggccacggacatccctgccagtgcctcgcctcccccagttgctggggtgcccttcttcaagcagtctccagggcaccagagccctcttgcctcccccaaagtgcccgtctgtcagcccttgaaggaagaagatgatgatgaggggccagtggacaagtcttccccaggaagtccccagagtccctctagtggggccgaggctgcagatgaggacagcaatgactcccctgcctccagctcctctaggcctcttaaggtgcggatcaagaccattaaaacatcctgcgggaatatcacaaggactgtaactcaggtcccctcagatcctgatccacctgcccccttggctgagggggccttcttggctgaggctagcctcttgaagctgtcccctgcaacacctacttctgagggtccaaaggtggtgagcgtacagttgggtgatggtacaaggctgaaaggcactgtgctgcctgtggccaccatccagaacgccagtactgccatgctgatggcagccagtgtggctcgcaaggctgtggtgctgcctggggggactgccaccagccctaagatgattgctaagaacgtgctaggcctggtgccccaagccctgcctaaggctgacgggcgggcagggctggggactgggggacagaaggtgaatggtgcctcggtggtgatggtgcaaccttcaaagacagctactgggccaagtacagggggcggcacagtgatatcacggacccagtccagcctggtggaggccttcaacaagatcctcaacagcaagaacctgctccctgcctataggccaaacctgagcccaccagctgaggctgggctggccctgcctcccaccggctaccgctgcctggagtgtggggatgccttctcattggagaagagcctggcacggcactatgaccgtcggagcatgcgcatcgaggtcacctgcaaccactgcgcccgccgcctggtcttcttcaacaagtgcagcctgctcctgcatgcacgtgaacacaaggacaaggggctcgtcatgcagtgctcacatttggtcatgaggcctgtagcccttgaccagatggtggggcagccggacatcacaccgctgctgcctgtagctgtcccacctgtctctggacctctggccttgcctgccttgggcaagggtgagggggccatcacctcctctgccattactacagttgctgctgaggcccctgtcctgccgctctccacagagccgcctgctgccctggccacctctgcttacacatgctttcgctgcctggagtgcaaggaacagtgccgggacaaggctggcatggcagctcacttccagcagctcggcccccctgcccctggggccaccagcaatgtgtgcccaacctgccccatgatgctccccaatcgctgcagcttcagcgcccaccagcgcatgcataagaatcgacccccccatgtctgtcctgagtgtgggggcaacttcctgcaagccaattttcagacccatctccgggaggcctgtctgcacgtctctcgccgtgtaggatacaggtgccccagctgttcagtggtgtttgggggtgtgaactccatcaagtcccacatccagacgtcgcactgcgaggttttccacaagtgccccatctgccccatggccttcaagtctgggccaagtgcccatgcccacctctactcccagcatcccagcttccaaactcagcaggccaagctgatctacaagtgcgccatgtgcgacacagtcttcactcacaaacccctcctctcctcacacttcgaccagcacttgctgccccagcgtgtcagtgtctttaagtgcccgtcttgtcctctgctctttgcccaaaaaaggaccatgctggaacatctcaagaacacccatcagtctgggcgcttggaggagactgctgggaaaggggccgggggtgccctgctgacccccaagactgagcctgaggagctggctgtttctcagggaggggcagcccctgctactgaggagtcgtcttcatcttcagaagaggaggaagtacccagctcccctgagcccccccgtccagccaaacggcctcggcgggaactagggagcaaaggcctcaagggtgggggtggggggcctggaggctggacctgtggcctgtgtcactcctggttccctgagcgtgatgaatacgtggcccacatgaagaaggagcatggcaagtcagtgaaaaagttcccctgtcgcctgtgtgagcgctccttctgctccgcccccagcctgaggcgccatgtcagagttaatcacgagggcatcaagcgagtttacccctgcaggtattgcacagagggaaaacgcaccttcagcagccgcctgatcctagagaaacatgtccaggtccggcacggcttgcagcttggggcccagtcccctggccgggggaccaccttggctcggggttccagtgccagagcccaggggccaggtcggaaacgccgccagtcttctgactcttgcagtgaggagcctgacagcacgacaccgccagccaagtcccccaggggcggacctggatctggaggccatggccctctgcgctaccggagcagcagctccacagaacagagcctcatgatggggttgagggtggaggatggtgcccagcagtgcctcgactgtggcttgtgctttgcctcccctggctccctgagccgacaccgtttcatcagccacaagaagagacggggtgtgggtaaagccagtgccctggggctgggggatggggaggaagaggcccctccatcaaggtctgaccccgatggtggagactcacccctgcctgcttctggaggcccactgacctgtaaggtctgtggcaagagctgcgacagccctctaaacctcaagacccacttccgcacgcatggcatggcgttcatcagggctcggcagggggctgttggggacaactag

SEQ ID NO: 4 - DNA, 3714 bp, Homo sapiens
atgggggatatgaagacccctgattttgatgacctccttgctgcctttgacatccctgacattgatgcgaatgaagccatccattctgggccagaagaaaatgaggggcctggaggcccagggaagccagaaccaggtgtaggaagtgaatctgaagacacagcagcagcctctgctggggatggccctggagttccagcccaggcctctgaccatggcctgccaccgccagacatttctgtagtcagtgtcattgtcaagaacactgtgtgtcccgagcagtctgaggccctggctggaggctcagcaggagacggggcccaggctgctggggtaactaaagaagggcctgtggggcctcatcgaatgcagaatggttttgggagccctgaaccttccctcccaggaactccccactctcctgctcctcccagtgggggcacctggaaagaaaaaggcatggaaggcaaaactcccttggacctgtttgctcattttggccctgagccaggggaccactcagatccgctgcctccctctgcaccctctcccactcgggagggggctctgaccccgcctcctttcccctcttcctttgagctggcccaggagaatggcccaggcatgcagccacctgtttcttccccaccattgggggccttgaagcaggagagctgcagcccccatcatccccaggtcctagcccaacaaggctcaggctccagccctaaggccacggacatccctgccagtgcctcgcctcccccagttgctggggtgcccttcttcaagcagtctccagggcaccagagccctcttgcctcccccaaagtgcccgtctgtcagcccttgaaggaagaagatgatgatgaggggccagtggacaagtcttccccaggaagtccccagagtccctctagtggggccgaggctgcagatgaggacagcaatgactcccctgcctccagctcctctaggcctcttaaggtgcggatcaagaccattaaaacatcctgcgggaatatcacaaggactgtaactcaggtcccctcagatcctgatccacctgcccccttggctgagggggccttcttggctgaggctagcctcttgaagctgtcccctgcaacacctacttctgagggtccaaaggtggtgagcgtacagttgggtgatggtacaaggctgaaaggcactgtgctgcctgtggccaccatccagaacgccagtactgccatgctgatggcagccagtgtggctcgcaaggctgtggtgctgcctggggggactgccaccagccctaagatgattgctaagaacgtgctaggcctggtgccccaagccctgcctaaggctgacgggcgggcagggctggggactgggggacagaaggtgaatggtgcctcggtggtgatggtgcaaccttcaaagacagctactgggccaagtacagggggcggcacagtgatatcacggacccagtccagcctggtggaggccttcaacaagatcctcaacagcaagaacctgctccctgcctataggccaaacctgagcccaccagctgaggctgggctggccctgcctcccaccggctaccgctgcctggagtgtggggatgccttctcattggagaagagcctggcacggcactatgaccgtcggagcatgcgcatcgaggtcacctgcaaccactgcgcccgccgcctggtcttcttcaacaagtgcagcctgctcctgcatgcacgtgaacacaaggacaaggggctcgtcatgcagtgctcacatttggtcatgaggcctgtagcccttgaccagatggtggggcagccggacatcacaccgctgctgcctgtagctgtcccacctgtctctggacctctggccttgcctgccttgggcaagggtgagggggccatcacctcctctgccattactacagttgctgctgaggcccctgtcctgccgctctccacagagccgcctgctgccccggccacctctgcttacacatgctttcgctgcctggagtgcaaggaacagtgccgggacaaggctggcatggcagctcacttccagcagctcggcccccctgcccctggggccaccagcaatgtgtgcccaacctgccccatgatgctccccaatcgctgcagcttcagcgcccaccagcgcatgcataagaatcgacccccccatgtctgtcctgagtgtgggggcaacttcctgcaagccaattttcagacccatctccgggaggcctgtctgcacgtctctcgccgtgtaggatacaggtgccccagctgttcagtggtgtttgggggtgtgaactccatcaagtcccacatccagacgtcgcactgcgaggttttccacaagtgccccatctgccccatggccttcaagtctgggccaagtgcccatgcccacctctactcccagcatcccagcttccaaactcagcaggccaagctgatctacaagtgcgccatgtgcgacacagtcttcactcacaaacccctcctctcctcacacttcgaccagcacttgctgccccagcgtgtcagtgtctttaagtgcccgtcttgtcctctgctctttgcccaaaaaaggaccatgctggaacatctcaagaacacccatcagtctgggcgcttggaggagactgctgggaaaggggccgggggtgccctgctgacccccaagactgagcctgaggagctggctgtttctcagggaggggcagcccctgctactgaggagtcgtcttcatcttcagaagaggaggaagtacccagctcccctgagcgcccccgtccagccaaacggcctcggcgggaactagggagcaaaggcctcaagggtgggggtggggggcctggaggctggacctgtggcctgtgtcactcctggttccctgagcgtgatgaatacgtggcccacatgaagaaggagcatggcaagtcagtgaaaaagttcccctgtcgcctgtgtgagcgctccttctgctccgcccccagcctgaggcgccatgtcagagttaatcacgagggcatcaagcgagtttacccctgcaggtattgcacagagggaaaacgcaccttcagcagccgcctgatcctagagaaacatgtccaggtccggcacggcttgcagcttggggcccagtcccctggccgggggaccaccttggctcggggttccagtgccagagcccaggggccaggtcggaaacgccgccagtcttctgactcttgcagtgaggagcctgacagcacgacaccgccagccaagtcccccaggggcggacctggatctggaggccatggccctctgcgctaccggagcagcagctccacagaacagagcctcatgatggggttgagggtggaggatggtgcccagcagtgcctcgactgtggcttgtgctttgcctcccctggctccctgagccgacaccgtttcatcagccacaagaagagacggggtgtgggtaaagccagtgccctggggctgggggatggggaggaagaggcccctccatcaaggtctgaccccgatggtggagactcacccctgcctgcttctggaggcccactgacctgtaaggtctgtggcaagagctgcgacagccctctaaacctcaagacccacttccgcacgcatggcatggcgttcatcagggctcggcagggggctgttggggacaactag

SEQ ID NO: 5 - protein, 1237 aa, Homo sapiens
MGDMKTPDFDDLLAAFDIPDIDANEAIHSGPEENEGPGGPGKPEPGVGSESEDTAAASAGDGPGVPAQASDHGLPPPDISVVSVIVKNTVCPEQSEALAGGSAGDGAQAAGVTKEGPVGPHRMQNGFGSPEPSLPGTPHSPAPPSGGTWKEKGMEGKTPLDLFAHFGPEPGDHSDPLPPSAPSPTREGALTPPPFPSSFELAQENGPGMQPPVSSPPLGALKQESCSPHHPQVLAQQGSGSIPKATDIPASASPPPVAGVPFFKQSPGHQSPLASPKVPVCQPLKEEDDDEGPVDKSSPGSPQSPSSGAEAADEDSNDSPASSSSRPLKVRIKTIKTSCGNITRTVTQVPSDPDPPAPLAEGAFLAEASLLKLSPATPTSEGPKVVSVQLGDGTRLKGTVLPVATIQNASTAMLMAASVARKAVVLPGGTATSPKMIAKNVLGLVPQALPKADGRAGLGTGGQKVNGASVVMVQPSKTATGPSTGGGTVISRTQSSLVEAFNKILNSKNLLPAYRPNLSPPAEAGLALPPTGYRCLECGDAFSLEKSLARHYDRRSMRIEVTCNHCARRLVFFNKCSLLLHAREHKDKGLVMQCSHLVMRPVALDQMVGQPDITPLLPVAVPPVSGPLALPALGKGEGAITSSAITTVAAEAPVLPLSTEPPAAPATSAYTCFRCLECKEQCRDKAGMAAHFQQLGPPAPGATSNVCPTCPMMLPNRCSFSAHQRMHKNRPPHVCPECGGNFLQANFQTHLREACLHVSRRVGYRCPSCSVVFGGVNSIKSHIQTSHCEVFHKCPICPMAFKSGPSAHAHLYSQHPSFQTQQAKLIYKCAMCDTVFTHKPLLSSHFDQHLLPQRVSVFKCPSCPLLFAQKRTMLEHLKNTHQSGRLEETAGKGAGGALLTPKTEPEELAVSQGGAAPATEESSSSSEEEEVPSSPEPPRPAKRPRRELGSKGLKGGGGGPGGWTCGLCHSWFPERDEYVAHMKKEHGKSVKKFPCRLCERSFCSAPSLRRHVRVNHEGIKRVYPCRYCTEGKRTFSSRLILEKHVQVRHGLQLGAQSPGRGTTLARGSSARAQGPGRKRRQSSDSCSEEPDSTTPPAKSPRGGPGSGGHGPLRYRSSSSTEQSLMMGLRVEDGAQQCLDCGLCFASPGSLSRHRFISHKKRRGVGKASALGLGDGEEEAPPSRSDPDGGDSPLPASGGPLTCKVCGKSCDSPLNLKTHFRTHGMAFIRARQGAVGDN

SEQ ID NO: 6 - protein, 1237 aa, Homo sapiens
MGDMKTPDFDDLLAAFDIPDIDANEAIHSGPEENEGPGGPGKPEPGVGSESEDTAAASAGDGPGVPAQASDHGLPPPDISVVSVIVKNTVCPEQSEALAGGSAGDGAQAAGVTKEGPVGPHRMQNGFGSPEPSLPGTPHSPAPPSGGTWKEKGMEGKTPLDLFAHFGPEPGDHSDPLPPSAPSPTREGALTPPPFPSSFELAQENGPGMQPPVSSPPLGALKQESCSPHHPQVLAQQGSGSSPKATDIPASASPPPVAGVPFFKQSPGHQSPLASPKVPVCQPLKEEDDDEGPVDKSSPGSPQSPSSGAEAADEDSNDSPASSSSRPLKVRIKTIKTSCGNITRTVTQVPSDPDPPAPLAEGAFLAEASLLKLSPATPTSEGPKVVSVQLGDGTRLKGTVLPVATIQNASTAMLMAASVARKAVVLPGGTATSPKMIAKNVLGLVPQALPKADGRAGLGTGGQKVNGASVVMVQPSKTATGPSTGGGTVISRTQSSLVEAFNKILNSKNLLPAYRPNLSPPAEAGLALPPTGYRCLECGDAFSLEKSLARHYDRRSMRIEVTCNHCARRLVFFNKCSLLLHAREHKDKGLVMQCSHLVMRPVALDQMVGQPDITPLLPVAVPPVSGPLALPALGKGEGAITSSAITTVAAEAPVLPLSTEPPAALATSAYTCFRCLECKEQCRDKAGMAAHFQQLGPPAPGATSNVCPTCPMMLPNRCSFSAHQRMHKNRPPHVCPECGGNFLQANFQTHLREACLHVSRRVGYRCPSCSVVFGGVNSIKSHIQTSHCEVFHKCPICPMAFKSGPSAHAHLYSQHPSFQTQQAKLIYKCAMCDTVFTHKPLLSSHFDQHLLPQRVSVFKCPSCPLLFAQKRTMLEHLKNTHQSGRLEETAGKGAGGALLTPKTEPEELAVSQGGAAPATEESSSSSEEEEVPSSPEPPRPAKRPRRELGSKGLKGGGGGPGGWTCGLCHSWFPERDEYVAHMKKEHGKSVKKFPCRLCERSFCSAPSLRRHVRVNHEGIKRVYPCRYCTEGKRTFSSRLILEKHVQVRHGLQLGAQSPGRGTTLARGSSARAQGPGRKRRQSSDSCSEEPDSTTPPAKSPRGGPGSGGHGPLRYRSSSSTEQSLMMGLRVEDGAQQCLDCGLCFASPGSLSRHRFISHKKRRGVGKASALGLGDGEEEAPPSRSDPDGGDSPLPASGGPLTCKVCGKSCDSPLNLKTHFRTHGMAFIRARQGAVGDN

SEQ ID NO: 7 - protein, 1237 aa, Homo sapiens
MGDMKTPDFDDLLAAFDIPDIDANEAIHSGPEENEGPGGPGKPEPGVGSESEDTAAASAGDGPGVPAQASDHGLPPPDISVVSVIVKNTVCPEQSEALAGGSAGDGAQAAGVTKEGPVGPHRMQNGFGSPEPSLPGTPHSPAPPSGGTWKEKGMEGKTPLDLFAHFGPEPGDHSDPLPPSAPSPTREGALTPPPFPSSFELAQENGPGMQPPVSSPPLGALKQESCSPHHPQVLAQQGSGSSPKATDIPASASPPPVAGVPFFKQSPGHQSPLASPKVPVCQPLKEEDDDEGPVDKSSPGSPQSPSSGAEAADEDSNDSPASSSSRPLKVRIKTIKTSCGNITRTVTQVPSDPDPPAPLAEGAFLAEASLLKLSPATPTSEGPKVVSVQLGDGTRLKGTVLPVATIQNASTAMLMAASVARKAVVLPGGTATSPKMIAKNVLGLVPQALPKADGRAGLGTGGQKVNGASVVMVQPSKTATGPSTGGGTVISRTQSSLVEAFNKILNSKNLLPAYRPNLSPPAEAGLALPPTGYRCLECGDAFSLEKSLARHYDRRSMRIEVTCNHCARRLVFFNKCSLLLHAREHKDKGLVMQCSHLVMRPVALDQMVGQPDITPLLPVAVPPVSGPLALPALGKGEGAITSSAITTVAAEAPVLPLSTEPPAAPATSAYTCFRCLECKEQCRDKAGMAAHFQQLGPPAPGATSNVCPTCPMMLPNRCSFSAHQRMHKNRPPHVCPECGGNFLQANFQTHLREACLHVSRRVGYRCPSCSVVFGGVNSIKSHIQTSHCEVFHKCPICPMAFKSGPSAHAHLYSQHPSFQTQQAKLIYKCAMCDTVFTHKPLLSSHFDQHLLPQRVSVFKCPSCPLLFAQKRTMLEHLKNTHQSGRLEETAGKGAGGALLTPKTEPEELAVSQGGAAPATEESSSSSEEEEVPSSPEPPRPAKRPRRELGSKGLKGGGGGPGGWTCGLCHSWFPERDEYVAHMKKEHGKSVKKFPCRLCERSFCSAPSLRRHVRVNHEGIKRVYPCRYCTEGKRTFSSRLILEKHVQVRHGLQLGAQSPGRGTTLARGSSARAQGPGRKRRQSSDSCSEEPDSTTPPAKSPRGGPGSGGHGPLRYRSSSSTEQSLMMGLRVEDGAQQCLDCGLCFASPGSLSRHRFISHKKRRGVGKASALGLGDGEEEAPPSRSDPDGGDSPLPASGGPLTCKVCGKSCDSPLNLKTHFRTHGMAFIRARQGAVGDN

SEQ ID NO: 8 - protein, 1237 aa, Homo sapiens; identical to SEQ ID NO: 7

SEQ ID NO: 9 - DNA, 20 bp, artificial sequence
  primer
cctcctcgttcctgttttca

SEQ ID NO: 10 - DNA, 20 bp, artificial sequence
  primer
caatggtggggaagaaacag

SEQ ID NO: 11 - DNA, 20 bp, artificial sequence
  primer
ggacctgtttgctcattttg

SEQ ID NO: 12 - DNA, 20 bp, artificial sequence
  primer
aactgtacgctcaccacctt

SEQ ID NO: 13 - DNA, 20 bp, artificial sequence
  primer
cgggaatatcacaaggactg

SEQ ID NO: 14 - DNA, 20 bp, artificial sequence
  primer
tataggcagggagcaggttc

SEQ ID NO: 15 - DNA, 20 bp, artificial sequence
  primer
aatggtgcctcggtggtgat

SEQ ID NO: 16 - DNA, 20 bp, artificial sequence
  primer
gagcatcatggggcaggttg

SEQ ID NO: 17 - DNA, 20 bp, artificial sequence
  primer
catgctttcgctgcctggag

SEQ ID NO: 18 - DNA, 20 bp, artificial sequence
  primer
gagggatatagcagggaaga

SEQ ID NO: 19 - DNA, 20 bp, artificial sequence
  primer
cctttctccaccctgctcat

SEQ ID NO: 20 - DNA, 20 bp, artificial sequence
  primer
acccattccccatccctctg

SEQ ID NO: 21 - DNA, 20 bp, artificial sequence
  primer
ccgtcttgtcctctgctctt

SEQ ID NO: 22 - DNA, 20 bp, artificial sequence
  primer
aggagagcaggggtagatgt

SEQ ID NO: 23 - DNA, 20 bp, artificial sequence
  primer
gcctggctctgacatctacc

SEQ ID NO: 24 - DNA, 20 bp, artificial sequence
  primer
ccttaccaccacccctactg

SEQ ID NO: 25 - DNA, 20 bp, artificial sequence
  primer
gctgtgctagggctttgagt

SEQ ID NO: 26 - DNA, 20 bp, artificial sequence
  primer
aggcagcagtagggaaaacc

SEQ ID NO: 27 - DNA, 20 bp, artificial sequence
  primer
aggagtcgtcttcatcttca

SEQ ID NO: 28 - DNA, 20 bp, artificial sequence
  primer
ttcactgacttgccatgctc

SEQ ID NO: 29 - DNA, 20 bp, artificial sequence
  primer
tggcgtcgtgattagtgatg

SEQ ID NO: 30 - DNA, 20 bp, artificial sequence
  primer
gcacacagagggctacaatg